(19) United States
(12) Patent Application Publication
(10) Pub. No.: US 2006/0246141 A1
(43) Pub. Date: Nov. 2, 2006

(54) Nanoparticulate lipase inhibitor formulations

(75) Inventors: Gary G. Liversidge, West Chester, PA (US); Scott Jenkins, Downingtown, PA (US)

Correspondence Address: ELAN DRUG DELIVERY, INC.;C/O FOLEY & LARDNER LLP, 3000 K STREET, N.W., SUITE 500, WASHINGTON, DC, 20007-5109, US

(73) Assignee: Elan Pharma International, Limited

(21) Appl. No.: 11/402,257

(22) Filed: Apr. 12, 2006

Related U.S. Application Data
(60) Provisional application No. 60/670,416, filed on Apr. 12, 2005.

(51) Int. Cl. A61K 31/365; A61K 9/14

(52) U.S. Cl. 424/489; 514/449; 977/906

(57) ABSTRACT

The invention relates to nanoparticulate lipase inhibitor compositions having improved pharmacokinetic profiles. The nanoparticulate lipase inhibitor compositions have an effective average particle size of less than about 2000 nm and are useful in the treatment of obesity and related diseases.

### CROSS-REFERENCE TO RELATED PATENT APPLICATIONS

This application claims benefit of U.S. Provisional Application No. 60/670,416, filed on Apr. 12, 2005.

### FIELD OF THE INVENTION

The invention relates to nanoparticulate lipase inhibitor compositions, including nanoparticulate orlistat formulations, with an average effective particle size of less than about 2000 nm. The invention also relates to methods of preparing nanoparticulate lipase inhibitors as well as to methods of treating patients using the nanoparticulate lipase inhibitor compositions.

### BACKGROUND OF THE INVENTION

### A. Background Regarding Lipase Inhibitors

During the past two decades, obesity has become a major health crisis in the western industrialized countries. For example, obesity among both adults and children has risen significantly in the U.S.: the latest data from the National Center for Health Statistics show that 30 percent of U.S. adults 20 years of age and older—over 60 million people—are obese. More disturbing is that over 9 million children (aged 6-19) in the U.S. are obese, this is more than triple the number of obese children noted in 1980. Obesity, which leads to other severe health conditions including diabetes, heart disease and cancer, is now the most common nutritional disorder in western industrialized nations.

The recent health crisis has spurred research in weight control, including studies in diet, exercise, surgery and pharmaceutical preparations to help reduce weight gain. Because obesity is caused by a build up of fat in the body due to, for example, the over-consumption of high fat foods, drug design strategies have included the blocking or stimulating of various biomolecules and enzymes involved in fat metabolism. Some strategies have encompassed serotonin and noradrenaline re-uptake inhibitors, β3-adrenoreceptor agonists, leptin agonists, melanocortin-3 agonists, cannabinoid (CB1) receptor blockers, and lipase inhibitors.

Lipase inhibitors have been recognized as an effective way to manage obesity by inhibiting the absorption of some dietary fats. Lipase is an enzyme that hydrolyzes lipids, the ester bonds in triglycerides, to form fatty acids and glycerol. Inhibitors of lipase, therefore, prevent the breakdown and subsequent use of the fatty acids and glycerols in the body.

1. Orlistat

Orlistat, also known as tetrahydroplipstatin (THL), is a reversible inhibitor of lipases. It exerts its therapeutic activity in the lumen of the stomach and small intestine by forming a covalent bond with the active serine residue site of gastric and pancreatic lipases. The inactivated enzymes are thus unavailable to hydrolyze dietary fat in the form of triglycerides into absorbable free fatty acids and monoglycerides. As undigested triglycerides are not absorbed, the resulting caloric deficit may have a positive effect on weight control. Systemic absorption of the drug is therefore not needed for activity. At the recommended therapeutic dose of 120 mg three times a day, orlistat inhibits dietary fat absorption by approximately 30%.

The chemical name of orlistat is (S)-2-formylamino-4-methyl-pentanoic acid (S)-1-[[(2S, 3S)-3-hexyl-4-oxo-2-oxetanyl]methyl]-dodecyl ester. Its empirical formula is C₂₉H₅₃NO₅, and its molecular weight is 495.7. It is a single diastereomeric molecule that contains four chiral centers, with a negative optical rotation in ethanol at 529 nm having the following structure:

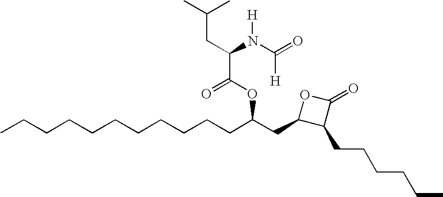

Orlistat is a white to off-white crystalline powder. Orlistat is practically insoluble in water, freely soluble in chloroform, and very soluble in methanol and ethanol. Orlistat has no pKa within the physiological pH range.

Orlistat is offered under the registered trademark XENICAL® by Hoffman-La Roche Inc. of Nutley, N.J. XENICAL® is available for oral administration in dark-blue, hard-gelatin capsules, with light-blue imprinting. Each capsule contains 120 mg of the active ingredient, orlistat. The capsules also contain the inactive ingredients microcrystalline cellulose, sodium starch glycolate, sodium lauryl sulfate, povidone, and talc. Each capsule shell contains gelatin, titanium dioxide, and FD&C Blue No. 1, with printing of pharmaceutical glaze NF, titanium dioxide, and FD&C Blue No. 1 aluminum lake.

The recommended dose of XENICAL® is one 120-mg capsule 3 times a day, once with each main meal containing fat (during or up to 1 hour after the meal).

Lipase inhibitors are described in, for example, U.S. Pat. No. 4,598,089 for “Leucine Derivatives” and 6,004,996 for “Tetrahydrolipstatin Containing Compositions.” U.S. Pat. No. 4,598,089 describes the orlistat compound, compositions for administration of an orlistat compound, and methods of treating obesity and hyperlipaemia in an afflicted mammal wherein orlistat is administered. U.S. Pat. No. 6,004,996 describes orlistat-containing particles having improved stability against moisture and heat during production and storage.

### B. Background Regarding Nanoparticulate Active Agent Compositions

Nanoparticulate active agent compositions, first described in U.S. Pat. No. 5,145,684 (“the '684 patent”), comprise particles of a poorly soluble therapeutic or diagnostic agent having adsorbed onto or associated with the surface thereof a non-crosslinked surface stabilizer. The '684 patent also describes methods of making such nanoparticulate active agent compositions but does not describe compositions comprising lipase inhibitors in nanoparticulate form. Methods of making nanoparticulate active agent compositions are described, for example, in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate active agent compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive Nanoparticulate Compositions Having Cationic Surface Stabilizers;” U.S. Pat. No. 6,431,478 for “Small Scale Mill;” U.S. Pat. No. 6,432,381 for “Methods for Targeting Drug Delivery to the Upper and/or Lower Gastrointestinal Tract;” U.S. Pat. No. 6,582,285 for “Apparatus for Sanitary Wet Milling;” and U.S. Pat. No. 6,592,903 for “Nanoparticulate Dispersions Comprising a Synergistic Combination of a Polymeric Surface Stabilizer and Dioctyl Sodium Sulfosuccinate;” U.S. Pat. No. 6,656,504 for “Nanoparticulate Compositions Comprising Amorphous Cyclosporine;” U.S. Pat. No. 6,742,734 for “System and Method for Milling Materials;” U.S. Pat. No. 6,745,962 for “Small Scale Mill and Method Thereof;” U.S. Pat. No. 6,811,767 for “Liquid droplet aerosols of nanoparticulate drugs;” U.S. Pat. No. 6,908,626 for “Compositions having a combination of immediate release and controlled release characteristics;” U.S. Pat. No. 6,969,529 for “Nanoparticulate compositions comprising copolymers of vinyl pyrrolidone and vinyl acetate as surface stabilizers;” U.S. Pat. No. 6,976,647 for “System and Method for Milling Materials;” and U.S. Pat. No. 6,991,191 for “Method of Using a Small Scale Mill;” all of which are specifically incorporated by reference. In addition, U.S. Patent Application No. 20020012675 A1, published on Jan. 31, 2002, for “Controlled Release Nanoparticulate Compositions,” describes nanoparticulate compositions and is specifically incorporated by reference. None of these references describe compositions of nanoparticulate lipase inhibitors.

Amorphous small particle compositions are described, for example, in U.S. Pat. No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter” all of which are specifically incorporated herein by reference.

There is a need for compositions of lipase inhibitors, such as orlistat, that have enhanced bioavailability, increased dissolution rate, reduced drug dosage and reduced adverse side effects. The present invention satisfies these needs.

### SUMMARY OF THE INVENTION

The present invention relates to stable nanoparticulate lipase inhibitor compositions, including lipase inhibitors such as orlistat, that are effective in the treatment of obesity and related diseases. The present invention also relates to methods of preparing such compositions, and to methods of treatment using such compositions.

In general, the compositions of the present invention include nanoparticulate lipase inhibitor particles and at least one surface stabilizer associated with or adsorbed to the surface of the nanoparticulate lipase inhibitor particles. The nanoparticulate lipase inhibitor particles have an effective average particles size of less than about 2000 nm.

In some embodiments, the lipase inhibitor is orlistat; the nanoparticulate orlistat particle may be in a crystalline phase, an amorphous phase, a semi-crystalline phase, a semi-amorphous phase, or a mixtures thereof.

Some compositions may include one or more surface stabilizers; for example, a composition may include at least one primary surface stabilizer and at least one secondary surface stabilizer. In some embodiments, a surface stabilizer may be a non-ionic surface stabilizer, an anionic surface stabilizer, a cationic surface stabilizer, a zwitterionic stabilizer or an ionic surface stabilizer. Stable nanoparticulate composition of the present invention may include particles of orlistat with one or more of dioctyl sodium sulfosuccinate, hypromellose and sodium lauryl sulfate associated with the surface of the particles.

Some compositions may include a nanoparticulate lipase inhibitor and one or more compounds (e.g., active agents) useful in treating obesity and related diseases (e.g., type II diabetes, high blood pressure, stroke, myocardial infarction, congestive heart failure, cancer, gallstones, gall bladder disease, gout, gouty arthritis, osteoarthritis, sleep apenea or pickwickian syndrome. Some compositions may also include pharmaceutically acceptable carriers, excipients or combinations thereof.

Another embodiment of the invention encompasses a lipase inhibitor, such as orlistat, composition, having equal efficacy at lower doses. For example, orlistat particles may possess an enhanced binding affinity for the lipase in the lumen of the stomach and small intestine as compared to conventional non-nanoparticulate orlistat compositions. Some embodiments allow for the administration of dosage amounts of less than about 375 mg daily of a nanoparticulate orlistat composition to have the same efficacy of dosage amounts of about 375 mg daily of a conventional non-nanoparticulate orlistat composition. Other embodiments allow for the administration of dosage amounts of less than about 360 mg daily of a nanoparticulate orlistat composition to have the same efficacy of a dosage amount of about 360 mg daily of a conventional non-nanoparticulate orlistat composition. Still other embodiments allow for the administration of dosage amounts of less than about 300 mg daily of a nanoparticulate orlistat composition to have the same efficacy of a dosage amount of about 300 mg daily of a conventional non-nanoparticulate orlistat composition. Some embodiments allow for the administration of dosage amounts of less than about 250 mg daily of a nanoparticulate orlistat composition to have the same efficacy of a dosage amount of about 250 mg daily of a conventional non-nanoparticulate orlistat composition. Other embodiments allow for the administration of dosage amounts of less than about 200 mg daily of a nanoparticulate orlistat composition to have the same efficacy of a dosage amount of about 200 mg daily of a conventional non-nanoparticulate orlistat composition.

The compositions of the invention may be formulated into any pharmaceutically acceptable dosage form. Formulations for administration may include but are not limited to solid dosage forms, liquid dosage forms, oral tablets, capsules, sachets, solutions, dispersions and mixtures thereof. Also contemplated are dosage forms such as liquid dispersions, gels, aerosols, ointments, creams, injectable formulations, controlled release formulations, fast melt formulations, lyophilized formulations, tablets, capsules, delayed release formulations, extended release formulations, pulsatile release formulations, mixed immediate release formulations, controlled release formulations, and mixtures thereof.

One aspect of the invention relates to pharmaceutical compositions comprising nanoparticulate lipase inhibitor compositions including, for example, orlistat. The pharmaceutical compositions include a nanoparticulate lipase inhibitor, at least one surface stabilizer, and a pharmaceutically acceptable carrier, as well as any desired excipients or a combination thereof.

The invention further discloses methods of preparing nanoparticulate lipase inhibitors such as those generally described above. Exemplary methods may include contacting a nanoparticulate lipase inhibitor such as orlistat and at least one surface stabilizer for a time under conditions sufficient to provide a nanoparticulate lipase inhibitor composition. The one or more surface stabilizers may be contacted with a nanoparticulate lipase inhibitor, either before, during, or after size reduction of the lipase inhibitor particle. The effective average particle size of the nanoparticulate lipase inhibitor is generally less than about 2000 nm. In some methods of preparing a nanoparticulate lipase inhibitor, the step of “contacting” may include one or more of the following: grinding, wet grinding, homogenization, freezing, and template emulsion. In other methods, “contacting” may include dissolving the lipase inhibitor particles in a solvent, adding at least one surface stabilizer thereto and precipitating the solubilized lipase inhibitor with the at least one surface stabilizer absorbed thereon or associated with the surface thereof by addition of a non-solvent. In some methods, the lipase inhibitor is orlistat, and the nanoparticulate orlistat particle is prepared as a crystalline phase, an amorphous phase, a semi-crystalline phase, a semi-amorphous phase, and mixtures thereof. In some methods, the compositions may include one or more of pharmaceutically acceptable excipients, carriers or combinations thereof; the compositions may also include one or more compounds (e.g., active agents) useful in treating obesity and related diseases. The compositions may include at least one primary surface stabilizer and at least one secondary stabilizer. In some methods the surface stabilizer may be a non-ionic surface stabilizer, an anionic surface stabilizer, a cationic surface stabilizer, a zwitterionic surface stabilizer, or an ionic surface stabilizer.

The present invention is also directed to methods of treatment including but not limited to obesity and related diseases, using the nanoparticulate lipase inhibitor, such as orlistat, compositions such as those generally described above. Such methods include administering to a subject a therapeutically effective amount of a nanoparticulate lipase inhibitor composition according to the invention. For example, a method of treatment may comprise administering an oral nanoparticulate lipase inhibitor, such as orlistat, where at least one surface stabilizer is associated with the surface of the orlistat particle and wherein the orlistat particles have an effective average particle size of less than about 2000 nm. In some methods, the lipase inhibitor, such as orlistat, may be administered in crystalline phase, an amorphous phase, a semi-crystalline phase, a semi-amorphous phase, or mixtures thereof; the dosage may be formulated for administration as oral tablets, capsules, sachets, solutions dispersions or mixtures thereof. The administered composition may comprise one or more pharmaceutically acceptable excipients, carriers, or a combination thereof. In some embodiments, the administered composition may comprise at least one primary and at least one secondary surface stabilizer; the surface stabilizer may be a non-ionic surface stabilizer, an anionic surface stabilizer, a cationic surface stabilizer, a zwitterionic surface stabilizer, or an ionic surface stabilizer. Other methods of treatment using the nanoparticulate compositions of the inventions are known to those of skill in the art.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

The present invention is directed to nanoparticulate compositions comprising a lipase inhibitor, such as orlistat. The compositions comprise a lipase inhibitor and preferably at least one surface stabilizer adsorbed on or associated with the surface of the lipase inhibitor particle. The lipase inhibitor, such as orlistat, particles have an effective average particle size of less than about 2000 nm.

As taught in the '684 patent, and as exemplified in the examples below, not every combination of surface stabilizer and active agent will result in a stable nanoparticulate composition. It was surprisingly discovered that stable, nanoparticulate lipase inhibitor formulations can be made.

Advantages of the nanoparticulate lipase inhibitor, such as orlistat, formulations of the invention include, but are not limited to: (1) smaller tablet or other solid dosage form size; (2) smaller doses of drug required to obtain the same pharmacological effect as compared to conventional microcrystalline or solubilized dosage forms of a lipase inhibitor; (3) an increased rate of dissolution for the lipase inhibitor compositions as compared to the conventional microcrystalline or solubilized dosage forms of the same lipase inhibitor; (4) the lipase inhibitor compositions can be used in conjunction with other active agents useful in treating obesity and related diseases; (5) increased bioavailability as compared to conventional forms of lipase inhibitors such as orlistat; (6) improved pharmacokinetic profiles; and (7) improved bioequivalency of the nanoparticulate lipase inhibitor compositions.

The present invention also includes a nanoparticulate lipase inhibitor, such as orlistat, compositions together with one or more non-toxic physiologically acceptable carriers, adjuvants, or vehicles, collectively referred to as carriers. The compositions can be formulated for oral administration in solid, liquid, or aerosol forms and the like.

A preferred dosage form of the invention is a solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

The present invention is described herein using several definitions as set forth below and throughout the application.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein with reference to particles of the lipase inhibitors, “stable” means that the lipase inhibitor particles do not appreciably flocculate or agglomerate due to interparticle attractive forces or otherwise spontaneously increase in particle size.

The term “effective average particle size of less than about 2000 nm” as used herein means that at least 50% of the lipase inhibitor particles have a size, by weight, of less than about 2000 nm, when measured by, for example, sedimentation field flow fractionation, photon correlation spectroscopy, light scattering, disk centrifugation, and other techniques known to those of skill in the art.

The term “conventional” or “non-nanoparticulate active agent” means an active agent which is solubilized or which has an effective average particle size of greater than about 2000 nm. Nanoparticulate active agents as defined herein have an effective average particle size of less than about 2000 nm.

As used herein, the phrase “therapeutically effective amount” shall mean the drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art. One of ordinary skill will appreciate that effective amounts of a drug can be determined empirically and can be employed in pure form or, where such form exists, in pharmaceutically acceptable salt, ester, or prodrug form.

The term “particulate” as used herein refers to a state of matter which is characterized by the presence of discrete particles, pellets, beads or granules irrespective of their size, shape or morphology. The term “multiparticulate” as used herein means a plurality of discrete, or aggregated, particles, pellets, beads, granules or mixture thereof irrespective of their size, shape or morphology.

A used herein the phrase “poorly water soluble drugs” refers to those drugs that have a solubility in water of less than about 30 mg/ml, preferably less than about 20 mg/ml, preferably less than about 10 mg/ml, or preferably less than about 1 mg/ml.

A. Preferred Characteristics of the Nanoparticulate Lipase Inhibitor Compositions of the Invention

1. Increased Efficacy and Bioavailability

The nanoparticulate lipase inhibitor, such as orlistat, formulations of the invention are proposed to exhibit increased efficacy and bioavailability at equal doses because more drug is dissolved while at the site of action as compared to a conventional formulation. This enables decreasing the total dose of the drug while maintaining equal efficacy.

2. Dissolution Profiles of the Lipase Inhibitor Compositions of the Invention

The nanoparticulate lipase inhibitor, such as orlistat, compositions of the invention are proposed to have unexpectedly dramatic dissolution profiles. Rapid dissolution of an administered active agent is preferable, as faster dissolution generally leads to faster onset of actions and enhanced efficacy. To improve the dissolution profile of the lipase inhibitor, it would be useful to increase the drug's dissolution so that it could attain a level close to 100%.

The lipase inhibitor compositions of the invention preferably have a dissolution profile in which within about 5 minutes at least about 20% of the composition is dissolved. In other embodiments of the invention, at least about 30% or at least about 40% of the lipase inhibitor composition is dissolved within about 5 minutes. In yet other embodiments of the invention, preferably at least about 40%, at least about 50%, at least about 60%, at least about 70%, or at least about 80% of the lipase inhibitor composition is dissolved within about 10 minutes. Finally, in another embodiment of the invention, preferably at least about 70%, at least about 80%, at least about 90%, or at least about 100% of the lipase inhibitor composition is dissolved within about 20 minutes.

Dissolution is preferably measured in a medium which is discriminating. Such a dissolution medium will produce two very different dissolution curves for two products having very different dissolution profiles in gastric juices, i.e., the dissolution medium is predictive of in vivo dissolution of a composition. An exemplary dissolution medium is an aqueous medium containing the surfactant sodium lauryl sulfate at 0.025 M. Determination of the amount of dissolved can be carried out by specrophotometry. The rotating blade methods (European Pharmacopoeia) can be used to measure dissolution.

3. Redispersability Profiles of the Lipase Inhibitor Compositions of the Invention

An additional feature of the lipase inhibitor, such as orlistat, compositions of the invention is that the compositions redisperse such that the effective average particle size of the redispersed lipase inhibitor particles is less than about 2 microns. This is significant, as if upon administration the lipase inhibitor compositions of the invention did not redisperse to a substantially nanoparticulate size, then the dosage form may lose the benefits afforded by formulating the lipase inhibitor into a nanoparticulate particle size.

This is because nanoparticulate active agent compositions benefit from the small particle size of the active agent; if the active agent does not disperse into the small particle sizes upon administration, then “clumps” or agglomerated active agent particles are formed, owing to the extremely high surface free energy of the nanoparticulate system and the thermodynamic driving force to achieve an overall reduction in free energy. With the formation of such agglomerated particles, the bioavailability of the dosage form may fall.

Moreover, the nanoparticulate lipase inhibitor compositions of the invention exhibit redispersion upon administration to a mammal, such as a human or animal, as demonstrated by reconstitution/redispersion in a biorelevant aqueous media such that the effective average particle size of the redispersed lipase inhibitor particles is less than about 2 microns. Such biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength. Such redispersion in a biorelevant media is predictive of in vivo efficacy of the lipase inhibitor dosage form.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 N, and NaCl solutions, ranging in concentration from about 0.001 to about 0.1 M, and mixtures thereof. For example, electrolyte solutions can be, but are not limited to, about 0.1 N HCl or less, about 0.01 N HCl or less, about 0.001 N HCl or less, about 0.1 M NaCl or less, about 0.01 M NaCl or less, about 0.001 M NaCl or less, and mixtures thereof. Of these electrolyte solutions, 0.01 N HCl and/or 0.1 M NaCl, are most representative of fasted human physiological conditions, owing to the pH and ionic strength conditions of the proximal gastrointestinal tract.

Electrolyte concentrations of 0.001 N HCl, 0.01 N HCl, and 0.1 N HCl correspond to pH 3, pH 2, and pH 1, respectively. Thus, a 0.01 N HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

Exemplary solutions of salts, acids, bases or combinations thereof, which exhibit the desired pH and ionic strength, include but are not limited to phosphoric acid/phosphate salts+sodium, potassium and calcium salts of chloride, acetic acid/acetate salts+sodium, potassium and calcium salts of chloride, carbonic acid/bicarbonate salts+sodium, potassium and calcium salts of chloride, and citric acid/citrate salts+sodium, potassium and calcium salts of chloride.

In other embodiments of the invention, the redispersed lipase inhibitor particles of the invention (redispersed in a biorelevant media, aqueous media, or any other appropriate media) have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. Such methods suitable for measuring effective average particle size are known to a person of ordinary skill in the art.

Redispersibility can be tested using any suitable means known in the art. See e.g., the example section of U.S. Pat. No. 6,375,986 for “Solid Dose Nanoparticulate Compositions Comprising a Synergistic Combination of a Polymeric Surface Stabilizer and Dioctyl Sodium Sulfosuccinate.”

4. The Pharmacokinetic Profiles of the Nanoparticulate Lipase Inhibitor Compositions of the Invention are not Affected by the Fed or Fasted State of the Subject Ingesting the Compositions

Although lipase inhibitors such as orlistat exert therapeutic activity in the lumen of the stomach and small intestine, therapeutic activity may also be desired in the circulatory system. Accordingly, the compositions of the invention encompass a nanoparticulate lipase inhibitor, such as orlistat, wherein the pharmacokinetic profile of the lipase inhibitor is not substantially affected by the fed or fasted state of a subject ingesting the composition. This means that there is little or no appreciable difference in the quantity of lipase inhibitor absorbed or the rate of drug absorption when the nanoparticulate compositions comprising a nanoparticulate lipase inhibitor, such as orlistat, is administered in the fed versus the fasted state.

Benefits of a dosage form which substantially eliminates the effect of food include an increase in subject convenience, thereby increasing subject compliance, as the subject does not need to ensure that they are taking a dose either with or without food. This is significant, as with poor subject compliance with a lipase inhibitor an increase in the medical condition for which the drug is being prescribed may be observed.

The invention also preferably provides compositions comprising at least one nanoparticulate lipase inhibitor such as orlistat, having a desirable pharmacokinetic profile when administered to mammalian subjects. The desirable pharmacokinetic profile of the compositions comprising at least one lipase inhibitor preferably includes, but is not limited to: (1) a Cₘₐₓ for the lipase inhibitor, such as orlistat, when assayed in the plasma of a mammalian subject following administration, that is preferably greater than the Cₘₐₓ for a non-nanoparticulate formulation of the same lipase inhibitor administered at the same dosage (e.g., XENICAL®); and/or (2) an AUC for the lipase inhibitor, such as orlistat, when assayed in the plasma of a mammalian subject following administration, that is preferably greater than the AUC for a non-nanoparticulate formulation of the same lipase inhibitor (e.g., XENICAL®), administered at the same dosage; and/or (3) a Tₘₐₓ for the lipase inhibitor, such as orlistat, when assayed in the plasma of a mammalian subject following administration, that is preferably less than the Tₘₐₓ for a non-nanoparticulate formulation of the same lipase inhibitor (e.g., XENICAL®), administered at the same dosage. The desirable pharmacokinetic profile, as used herein, is the pharmacokinetic profile measured after the initial dose of the lipase inhibitor.

In one embodiment, a composition comprising at least one nanoparticulate lipase inhibitor, such as orlistat, exhibits in comparative pharmacokinetic testing with a non-nanoparticulate formulation of the same lipase inhibitor (e.g., XENICAL®), administered at the same dosage, a Tₘₐₓ not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ exhibited by the non-nanoparticulate lipase inhibitor formulation.

In another embodiment, the composition comprising at least one nanoparticulate lipase inhibitor, such as orlistat, exhibits in comparative pharmacokinetic testing with a non-nanoparticulate formulation of the same lipase inhibitor (e.g., XENICAL®), administered at the same dosage, a Cₘₐₓ which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by the non-nanoparticulate lipase inhibitor formulation.

In yet another embodiment, the composition comprising at least one nanoparticulate lipase inhibitor, such as orlistat, exhibits in comparative pharmacokinetic testing with a non-nanoparticulate formulation of the same lipase inhibitor (e.g., XENICAL®), administered at the same dosage, an AUC which is at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate lipase inhibitor formulation.

5. Bioequivalency of the Lipase Inhibitor Compositions of the Invention when Administered in the Fed Versus the Fasted State

The invention also encompasses a composition comprising at least one nanoparticulate lipase inhibitor, such as orlistat, in which administration of the composition to a subject in a fasted state is bioequivalent to administration of the composition to a subject in a fed state. The difference in absorption (e.g., AUC), difference in Cₘₐₓ, or the difference in AUC and Cₘₐₓ, for the compositions comprising the nanoparticulate lipase inhibitor when administered in the fed versus the fasted state, is preferably less than about 60%, less than about 55%, less than about 50%, less than about 45%, less than about 40%, less than about 35%, less than about 30%, less than about 25%, less than about 20%, less than about 15%, less than about 10%, less than about 5%, or less than about 3%.

In one embodiment of the invention, the invention encompasses compositions comprising at least one nanoparticulate lipase inhibitor, such as orlistat, wherein administration of the composition to a subject in a fasted state is bioequivalent to administration of the composition to a subject in a fed state, in particular as defined by Cₘₐₓ and AUC guidelines given by the U.S. Food and Drug Administration and the corresponding European regulatory agency (EMEA). Under U.S. FDA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). To show bioequivalency between two compounds or administration conditions pursuant to Europe's EMEA guidelines, the 90% CI for AUC must be between 0.80 to 1.25 and the 90% CI for Cₘₐₓ must between 0.70 to 1.43.

6. Lipase Inhibitor Compositions Used in Conjunction with Other Active Agents

The lipase inhibitor, such as orlistat, compositions and methods of the invention can additionally comprise one or more compounds useful in treating obesity and related diseases, or the lipase inhibitor compounds can be administered in conjunction with such a compound. Examples of such compounds include anti-obesity agents, appetite suppressants, anti-diabetic agents, anti-hyperlipidemia agents, hypolipidemic agents, hypocholesterolemic agents, lipid-modulating agents, cholesterol-lowering agents, lipid-lowering agents, anti-hypertensive agents, agents used to treat sleep disorders, agents used to treat substance abuse and addictive disorders, anti-anxiety agents, anti-depressants, anti-psychotic agents, cognition enhancing agents, agents used to treat cognitive disorders, agents used to treat Alzheimer's disease, agents used to treat Parkinson's disease, anti-inflammatory agents, agents used to treat neurodegeneration, agents used to treat arteriosclerosis, agents used to treat respiratory conditions, agents used to treat bowel disorders, cardiac glycosides, and anti-tumor agents.

B. Compositions

The invention provides compositions and methods for making compositions comprising lipase inhibitor particles and at least one surface stabilizer. The surface stabilizers preferably are adsorbed on, or associated with, the surface of the lipase inhibitor particles. The surface stabilizers physically adhere on, or associate with, the surface of the nanoparticulate lipase inhibitor particles, but do not chemically react with the lipase inhibitor particles or itself. Individually absorbed molecules of the surface stabilizer are essentially free of intermolecular cross-linkages.

The present invention also includes lipase inhibitor compositions together with one or more non-toxic physiologically acceptable carriers, adjuvants, or vehicles, collectively referred to as carriers. The compositions can be formulated for parenteral injection (e.g., for intravenous, intramuscular, or subcutaneous), oral administration in solid, liquid, or aerosol form, vaginal, nasal, rectal, otic, ocular, local (powders, ointments or drops), buccal, intracistemal, intraperitoneal, or topical administration, and the like.

1. Lipase Inhibitor

Any suitable lipase inhibitor can be utilized in the methods and compositions of the invention. An exemplary lipase inhibitor is orlistat.

2. Surface Stabilizers

The choice of a surface stabilizer for a lipase inhibitor, such as orlistat, is non-trivial and required extensive experimentation to realize a desirable formulation. Accordingly, the present invention is directed to the surprising discovery that nanoparticulate lipase inhibitor compositions can be made.

Combinations of more than one surface stabilizers can be used in the invention. For example, a composition may include at least one primary surface stabilizer and at least one secondary surface stabilizer. Useful surface stabilizers which can be employed in the invention include, but are not limited to, known organic and inorganic pharmaceutical excipients. Such excipients include various polymers, low molecular weight oligomers, natural products, and surfactants. Surface stabilizers include nonionic, anionic, cationic, ionic, and zwitterionic surfactants.

Representative examples of surface stabilizers include hydroxypropyl methylcellulose (now known as hypromellose), hydroxypropylcellulose, diocytl sodium culfoccunate, polyvinylpyrrolidone, sodium lauryl sulfate, dioctyl sodium sulfosuccinate (also known as docusate sodium), gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-lOG® or Surfactant 10-G® (Olin Chemicals, Stamford, Conn.); Crodestas SL-40® (Croda, Inc.); and SA90HCO, which is C₁₈H₃₇CH₂(CON(CH₃)—CH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)4 ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:

(i) none of R₁-R₄ are CH₃;

(ii) one of R₁-R₄ is CH₃;

(iii) three of R₁-R₄ are CH₃;

(iv) all of R₁-R₄ are CH₃;

(v) two of R₁-R₄ are CH₃, one of R₁-R₄ is C₆H₅CH₂, and one of R₁-R₄ is an alkyl chain of seven carbon atoms or less;

(vi) two of R₁-R₄ are CH₃, one of R₁-R₄ is C₆H₅CH₂, and one of R₁-R₄ is an alkyl chain of nineteen carbon atoms or more;

(vii) two of R₁-R₄ are CH₃ and one of R₁-R₄ is the group C₆H₅(CH₂)ₙ, where n>1;

(viii) two of R₁-R₄ are CH₃, one of R₁-R₄ is C₆H₅CH₂, and one of R₁-R₄ comprises at least one heteroatom;

(ix) two of R₁-R₄ are CH₃, one of R₁-R₄ is C₆H₅CH₂, and one of R₁-R₄ comprises at least one halogen;

(x) two of R₁-R₄ are CH₃, one of R₁-R₄ is C₆H₅CH₂, and one of R₁-R₄ comprises at least one cyclic fragment;

(xi) two of R₁-R₄ are CH₃ and one of R₁-R₄ is a phenyl ring; or

(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The surface stabilizers are commercially available and/or can be prepared by techniques known in the art. Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), specifically incorporated by reference.

a. Povidone Polymers

Povidone polymers are exemplary surface stabilizers for use in formulating an injectable nanoparticulate lipase inhibitor composition. Povidone polymers, also known as polyvidon(e), povidonum, PVP, and polyvinylpyrrolidone, are sold under the trade names Kollidon® (BASF Corp.) and Plasdone® (ISP Technologies, Inc.). They are polydisperse macromolecular molecules, with a chemical name of 1-ethenyl-2-pyrrolidinone polymers and 1-vinyl-2-pyrrolidinone polymers. Povidone polymers are produced commercially as a series of products having mean molecular weights ranging from about 10,000 to about 700,000 daltons. To be useful as a surface modifier for a drug compound to be administered to a mammal, the povidone polymer must have a molecular weight of less than about 40,000 daltons, as a molecular weight of greater than 40,000 daltons would have difficulty clearing the body.

Povidone polymers are prepared by, for example, Reppe's process, comprising: (1) obtaining 1,4-butanediol from acetylene and formaldehyde by the Reppe butadiene synthesis; (2) dehydrogenating the 1,4-butanediol over copper at 200° to form γ-butyrolactone; and (3) reacting γ-butyrolactone with ammonia to yield pyrrolidone. Subsequent treatment with acetylene gives the vinyl pyrrolidone monomer. Polymerization is carried out by heating in the presence of H₂O and NH₃. See The Merck Index, 10th Edition, pp. 7581 (Merck & Co., Rahway, N.J., 1983).

The manufacturing process for povidone polymers produces polymers containing molecules of unequal chain length, and thus different molecular weights. The molecular weights of the molecules vary about a mean or average for each particular commercially available grade. Because it is difficult to determine the polymer's molecular weight directly, the most widely used method of classifying various molecular weight grades is by K-values, based on viscosity measurements. The K-values of various grades of povidone polymers represent a function of the average molecular weight, and are derived from viscosity measurements and calculated according to Fikentscher's formula.

The weight-average of the molecular weight, Mw, is determined by methods that measure the weights of the individual molecules, such as by light scattering.

TABLE 1
Povidone data

| | | Mv | Mw | Mn |
|---|---|---|---|---|
| Povidone | K-Value | (Daltons)** | (Daltons)** | (Daltons)** |
| Plasdone | 17 ± 1 | 7,000 | 10,500 | 3,000 |
| C-15 ® | | | | |
| Plasdone | 30.5 ± 1.5 | 38,000 | 62,500* | 16,500 |
| C-30 ® | | | | |
| Kollidon 12 | 11-14 | 3,900 | 2,000-3,000 | 1,300 |
| PF ® | | | | |
| Kollidon 17 | 16-18 | 9,300 | 7,000-11,000 | 2,500 |
| PF ® | | | | |
| Kollidon 25 ® | 24-32 | 25,700 | 28,000-34,000 | 6,000 |

*Because the molecular weight is greater than 40,000 daltons, this povidone polymer is not useful as a surface stabilizer for a drug compound to be administered parenterally (i.e., injected).
**Mv is the viscosity-average molecular weight; Mn is the number-average molecular weight, and Mw is the weight average molecular weight. Mw and Mn were determined by light scattering and ultra-centrifugation, and Mv was determined by viscosity measurements.

3. Other Pharmaceutical Excipients

Pharmaceutical compositions according to the invention may also comprise one or more binding agents, filling agents, penetration enhancers, lubricating agents, suspending agents, sweeteners, flavoring agents, preservatives, buffers, wetting agents, disintegrants, effervescent agents, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil® 200, talc, stearic acid, magnesium stearate, calcium stearate, and silica gel.

Aqueous suspensions comprising the nanoparticulate lipase inhititors can be in admixture with excipients suitable for the manufacture of aqueous suspensions. Such excipients are suspending agents, for example, sodium carboxymethylcellulose, methylcellulose, hydroxy-propylmethylcellulose, sodium alginate, polyvinylpyrrolidone, gum tragacanth and gum acadia.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Examples of buffers are phosphate buffers, citrate buffers and buffers made from other organic acids.

Examples of wetting or dispersing agents are a naturally-occurring phosphatide, for example, lecithin or condensation products of n-alkylene oxide with fatty acids, for example, polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol mono-oleate, or condensation products of ethylene oxide with partial esters derived from fatty acids and hexitol anhydrides, for example, polyethylene sorbitan monooleate.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

4. Enhancers

The lipase inhibitor component of the composition may be accompanied, for example, by an enhancer compound to modify the bioavailability or therapeutic effect of the active ingredient. As used herein, the term “enhancer” refers to a compound which is capable of enhancing the absorption and/or bioavailability of an active ingredient by promoting net transport across the gastro-intestinal tract in an animal, such as a human. Enhancers include but are not limited to medium chain fatty acids; salts, esters, ethers and derivatives thereof, including glycerides and triglycerides; non-ionic surfactants such as those that can be prepared by reacting ethylene oxide with a fatty acid, a fatty alcohol, an alkylphenol or a sorbitan or glycerol fatty acid ester; cytochrome P450 inhibitors, P-glycoprotein inhibitors and the like; and mixtures of two or more of these agents.

5. Nanoparticulate Lipase Inhibitor Particle Size

As used herein, particle size is determined on the basis of the weight average particle size as measured by conventional particle size measuring techniques well known to those skilled in the art. Such techniques include, for example, sedimentation field flow fractionation, photon correlation spectroscopy, light scattering, and disk centrifugation.

The compositions of the invention comprise at least one lipase inhibitor having an effective average particle size of less than about 2000 nm (i.e., 2 microns). In other embodiments of the invention, the lipase inhibitor nanoparticles have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

An “effective average particle size of less than about 2000 nm” means that at least 50% of the lipase inhibitor particles have a particle size less than the effective average, by weight, i.e., less than about 2000 nm. If the “effective average particle size” is less than about 1900 nm, then at least about 50% of the lipase inhibitor particles have a size of less than about 1900 nm, when measured by the above-noted techniques. The same is true for the other particle sizes referenced above. In other embodiments, at least about 60%, at least about 70%, at least about 80%, at least about 90%, at least about 95%, or at least about 99% of the lipase inhibitor particles have a particle size less than the effective average, i.e., less than about 2000 nm, less than about 1900 nm, less than about 1800 nm, etc.

In the invention, the value for D50 of a nanoparticulate lipase inhibitor composition is the particle size below which 50% of the lipase inhibitor particles fall, by weight. Similarly, D90 is the particle size below which 90% of the lipase inhibitor particles fall, by weight.

6. Concentration of Lipase Inhibitor and Surface Stabilizers

The relative amounts of lipase inhibitor, such as orlistat, and one or more surface stabilizers can vary widely. The optimal amount of the individual components can depend, for example, upon the particular lipase inhibitor selected, the hydrophilic lipophilic balance (HLB), melting point, and the surface tension of water solutions of the stabilizer, etc.

The concentration of the lipase inhibitor can vary from about 99.5% to about 0.001%, from about 95% to about 0.1%, or from about 90% to about 0.5%, by weight, based on the total combined weight of the lipase inhibitor and at least one surface stabilizer, not including other excipients.

The concentration of the at least one surface stabilizer can vary from about 0.5% to about 99.999%, from about 5.0% to about 99.9%, or from about 10% to about 99.5%, by weight, based on the total combined dry weight of the lipase inhibitor and at least one surface stabilizer, not including other excipients.

7. Exemplary Nanoparticulate Orlistat Tablet Formulations

Several exemplary orlistat tablet formulations are given below. These examples are not intended to limit the claims in any respect, but rather to provide exemplary tablet formulations of orlistat which can be utilized in the methods of the invention. Such exemplary tablets can also comprise a coating agent.

TABLE 2
Exemplary Nanoparticulate
Orlistat Tablet Formulation #1

| Component. | g/Kg |
|---|---|
| Orlistat | about 50 to about 500 |
| Hypromellose, USP | about 10 to about 70 |
| Docusate Sodium, USP | about 1 to about 10 |
| Sucrose, NF | about 100 to about 500 |
| Sodium Lauryl Sulfate, NF | about 1 to about 40 |
| Lactose Monohydrate, NF | about 50 to about 400 |
| Silicified Microcrystalline Cellulose | about 50 to about 300 |
| Crospovidone, NF | about 20 to about 300 |
| Magnesium Stearate, NF | about 0.5 to about 5 |

TABLE 3
Exemplary Nanoparticulate
Orlistat Tablet Formulation #2

| Component | g/Kg |
|---|---|
| Orlistat | about 100 to about 300 |
| Hypromellose, USP | about 30 to about 50 |
| Docusate Sodium, USP | about 0.5 to about 10 |
| Sucrose, NF | about 100 to about 300 |
| Sodium Lauryl Sulfate, NF | about 1 to about 30 |
| Lactose Monohydrate, NF | about 100 to about 300 |
| Silicified Microcrystalline Cellulose | about 50 to about 200 |
| Crospovidone, NF | about 50 to about 200 |
| Magnesium Stearate, NF | about 0.5 to about 5 |

TABLE 4
Exemplary Nanoparticulate
Orlistat Tablet Formulation #3

| Component | g/Kg |
|---|---|
| Orlistat | about 200 to about 225 |
| Hypromellose, USP | about 42 to about 46 |
| Docusate Sodium, USP | about 2 to about 6 |
| Sucrose, NF | about 200 to about 225 |
| Sodium Lauryl Sulfate, NF | about 12 to about 18 |
| Lactose Monohydrate, NF | about 200 to about 205 |
| Silicified Microcrystalline Cellulose | about 130 to about 135 |
| Crospovidone, NF | about 112 to about 118 |
| Magnesium Stearate, NF | about 0.5 to about 3 |

TABLE 5
Exemplary Nanoparticulate
Orlistat Tablet Formulation #4

| Component | g/Kg |
|---|---|
| Orlistat | about 119 to about 224 |
| Hypromellose, USP | about 42 to about 46 |
| Docusate Sodium, USP | about 2 to about 6 |
| Sucrose, NF | about 119 to about 224 |
| Sodium Lauryl Sulfate, NF | about 12 to about 18 |
| Lactose Monohydrate, NF | about 119 to about 224 |
| Silicified Microcrystalline Cellulose | about 129 to about 134 |
| Crospovidone, NF | about 112 to about 118 |
| Magnesium Stearate, NF | about 0.5 to about 3 |

C. Methods of Making Nanoparticulate Lipase Inhibitor Compositions

The nanoparticulate lipase inhibitor, such as orlistat, compositions can be made using any suitable method known in the art, for example, milling, homogenization, precipitation, freezing, supercritical fluid particle generation techniques, or template emulsion techniques. Exemplary methods of making nanoparticulate active agent compositions are described in the '684 patent. Methods of making nanoparticulate active agent compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference.

The resultant nanoparticulate lipase inhibitor compositions or dispersions can be utilized in solid or liquid dosage formulations, such as liquid dispersions, gels, aerosols, ointments, creams, controlled release formulations, fast melt formulations, lyophilized formulations, tablets, capsules, delayed release formulations, extended release formulations, pulsatile release formulations, mixed immediate release and controlled release formulations, etc.

An exemplary method of preparing the nanoparticulate lipase inhibitor, such as orlistat, formulations of the invention comprises the steps of: (1) dispersing the desired dosage amount of a lipase inhibitor in a liquid dispersion media in which the drug is poorly soluble; and (2) mechanically reducing the particle size of the lipase inhibitor to an effective average particle size of less than about 2000 nm. A surface stabilizer can be added to the dispersion media either before, during, or after particle size reduction of the lipase inhibitor. The liquid dispersion medium can be maintained at a physiologic pH, for example, within the range of from about 3.0 to about 8.0 during the size reduction process; more preferably within the range of from about 5.0 to about 7.5 during the size reduction process. Preferably, the dispersion media used for the size reduction process is aqueous, although any dispersion media in which the lipase inhibitor is poorly soluble can be used, such as safflower oil, ethanol, t-butanol, glycerin, polyethylene glycol (PEG), hexane, or glycol.

Using a particle size reduction method, the particle size of the lipase inhibitor is reduced to an effective average particle size of less than about 2000 nm. Effective methods of providing mechanical force for particle size reduction of the lipase inhibitor include ball milling, media milling, and homogenization, for example, with a Microfluidizer® (Microfluidics Corp.).

1. Lipase Inhibitor Particle Size Reduction Using Milling

Milling a lipase inhibitor, such as orlistat, to obtain a nanoparticulate dispersion comprises dispersing the lipase inhibitor particles in a liquid dispersion medium in which the lipase inhibitor is poorly soluble, followed by applying mechanical means in the presence of grinding media to reduce the particle size of the lipase inhibitor to the desired effective average particle size. The dispersion medium can be, for example, water, safflower oil, ethanol, t-butanol, glycerin, polyethylene glycol (PEG), hexane, or glycol. A preferred dispersion medium is water.

The lipase inhibitor particles can be reduced in size in the presence of at least one surface stabilizer. Alternatively, the lipase inhibitor particles can be contacted with one or more surface stabilizers after attrition. Other compounds, such as a diluent, can be added to the lipase inhibitor/surface stabilizer composition during the size reduction process. Dispersions can be manufactured continuously or in a batch mode.

The lipase inhibitor can be added to a liquid media in which it is essentially insoluble to form a premix. The surface stabilizer can be present in the premix or it can be added to the lipase inhibitor dispersion following particle size reduction. The premix can be used directly by subjecting it to mechanical means to reduce the average lipase inhibitor particle size in the dispersion to less than about 2000 nm. It is preferred that the premix be used directly when a ball mill is used for attrition. Alternatively, the lipase inhibitor and at least one surface stabilizer can be dispersed in the liquid media using suitable agitation, e.g., a Cowles type mixer, until a homogeneous dispersion is observed in which there are no large agglomerates visible to the naked eye. It is preferred that the premix be subjected to such a pre-milling dispersion step when a re-circulating media mill is used for attrition.

The mechanical means applied to reduce the lipase inhibitor particle size can take the form of a dispersion mill. Suitable dispersion mills include a ball mill, an attritor mill, a vibratory mill, and media mills such as a sand mill and a bead mill. A media mill is preferred due to the relatively shorter milling time required to provide the desired reduction in particle size. For media milling, the apparent viscosity of the premix is preferably from about 100 to about 1000 centipoise, and for ball milling the apparent viscosity of the premix is preferably from about 1 up to about 100 centipoise. Such ranges tend to afford an optimal balance between efficient particle size reduction and media erosion.

Ball milling is a low energy milling process that uses milling media, drug, stabilizer, and liquid. The materials are placed in a milling vessel that is rotated at optimal speed such that the media cascades and reduces the drug particle size by impaction. The media used must have a high density as the energy for the particle reduction is provided by gravity and the mass of the attrition media.

Media milling is a high energy milling process. Lipase inhibitor, such as orlistat, surface stabilizer, and liquid are placed in a reservoir and re-circulated in a chamber comprising grinding media and a rotating shaft/impeller. The rotating shaft agitates the grinding media which subjects the lipase inhibitor to impaction and sheer forces, thereby reducing the lipase inhibitor particle size.

The attrition time can vary widely and depends primarily upon the particular mechanical means and processing conditions selected. For ball mills, processing times of up to five days or longer may be required. Alternatively, processing times of less than 1 day (residence times of one minute up to several hours) are possible with the use of a high shear media mill.

The lipase inhibitor particles can be reduced in size at a temperature which does not significantly degrade the lipase inhibitor molecule. Processing temperatures of less than about 30° C. to less than about 40° C. are ordinarily preferred. If desired, the processing equipment can be cooled with conventional cooling equipment. Control of the temperature, e.g., by jacketing or immersion of the milling chamber in ice water, is contemplated. Generally, the method of the invention is conveniently carried out under conditions of ambient temperature and at processing pressures which are safe and effective for the milling process. Ambient processing pressures are typical of ball mills, attritor mills, and vibratory mills.

Grinding Media

The grinding media for the particle size reduction step can be selected from rigid media preferably spherical or particulate in form having an average size less than about 3 mm and, more preferably, less than about 1 mm. Such media desirably can provide the particles of the invention with shorter processing times and impart less wear to the milling equipment. The selection of material for the grinding media is not believed to be critical. Zirconium oxide, such as 95% ZrO stabilized with magnesia, zirconium silicate, ceramic, stainless steel, titania, alumina, 95% ZrO stabilized with yttrium, glass grinding media, and polymeric grinding media are exemplary grinding materials.

The grinding media can comprise particles that are preferably substantially spherical in shape, e.g., beads, consisting essentially of polymeric resin or other suitable material. Alternatively, the grinding media can comprise a core having a coating of a polymeric resin adhered thereon. The polymeric resin can have a density from about 0.8 to about 3.0 g/cm³.

In general, suitable polymeric resins are chemically and physically inert, substantially free of metals, solvent, and monomers, and of sufficient hardness and friability to enable them to avoid being chipped or crushed during grinding. Suitable polymeric resins include crosslinked polystyrenes, such as polystyrene crosslinked with divinylbenzene; styrene copolymers; polycarbonates; polyacetals, such as Delrin® (E.I. du Pont de Nemours and Co.); vinyl chloride polymers and copolymers; polyurethanes; polyamides; poly(tetrafluoroethylenes), e.g., Teflon® (E.I. du Pont de Nemours and Co.), and other fluoropolymers; high density polyethylenes; polypropylenes; cellulose ethers and esters such as cellulose acetate; polyhydroxymethacrylate; polyhydroxyethyl acrylate; and silicone-containing polymers such as polysiloxanes and the like. The polymer can be biodegradable. Exemplary biodegradable polymers include poly(lactides), poly(glycolide) copolymers of lactides and glycolide, polyanhydrides, poly(hydroxyethyl methacylate), poly(imino carbonates), poly(N-acylhydroxyproline)esters, poly(N-palmitoyl hydroxyproline) esters, ethylene-vinyl acetate copolymers, poly(orthoesters), poly(caprolactones), and poly(phosphazenes). For biodegradable polymers, contamination from the media itself advantageously can metabolize in vivo into biologically acceptable products that can be eliminated from the body.

The grinding media preferably ranges in size from about 0.01 to about 3 mm. For fine grinding, the grinding media is preferably from about 0.02 to about 2 mm, and more preferably from about 0.03 to about 1 mm in size.

In a preferred grinding process the lipase inhibitor, such as orlistat, particles are made continuously. Such a method comprises continuously introducing the lipase inhibitor into a milling chamber, contacting the compounds with grinding media while in the chamber to reduce the particle size, and continuously removing the nanoparticulate lipase inhibitor from the milling chamber.

The grinding media is separated from the milled nanoparticulate lipase inhibitor using conventional separation techniques, in a secondary process such as by simple filtration, sieving through a mesh filter or screen, and the like.

Sterile Product Manufacturing

Development of injectable compositions requires the production of a sterile product. The manufacturing process of the present invention is similar to typical known manufacturing processes for sterile suspensions. A typical sterile suspension manufacturing process flowchart is as follows:

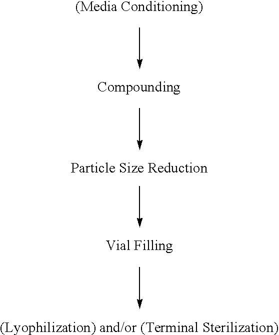

As indicated by the optional steps in parentheses, some of the processing is dependent upon the method of particle size reduction and/or method of sterilization. For example, media conditioning is not required for a milling method that does not use media. If terminal sterilization is not feasible due to chemical and/or physical instability, aseptic processing can be used.

2. Precipitation to Obtain Nanoparticulate Lipase Inhibitor Compositions

Another method of forming the desired nanoparticulate lipase inhibitor, such as orlistat, composition is by microprecipitation. This is a method of preparing stable dispersions of poorly soluble active agents in the presence of one or more surface stabilizers and one or more colloid stability enhancing surface active agents free of any trace toxic solvents or solubilized heavy metal impurities. Such a method comprises, for example: (1) dissolving the lipase inhibitor in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

3. Homogenization to Obtain Nanoparticulate Lipase Inhibitor Compositions

Exemplary homogenization methods of preparing nanoparticulate active agent compositions are described in U.S. Pat. No. 5,510,118, for “Process of Preparing Therapeutic Compositions Containing Nanoparticles”. Such a method comprises dispersing particles of a lipase inhibitor, such as orlistat, in a liquid dispersion medium, followed by subjecting the dispersion to homogenization to reduce the particle size of a lipase inhibitor to the desired effective average particle size. The lipase inhibitor particles can be reduced in size in the presence of at least one surface stabilizer. Alternatively, the lipase inhibitor particles can be contacted with one or more surface stabilizers either before or after attrition. Other compounds, such as a diluent, can be added to the lipase inhibitor/surface stabilizer composition either before, during, or after the size reduction process. Dispersions can be manufactured continuously or in a batch mode.

An exemplary homogenization process is described using the Microfluidizer® (discussed in U.S. Pat. No. 5,510,118). Lipase inhibitor, such as orlistat, surface stabilizer, and liquid (or drug and liquid with the surface stabilizer added after particle size reduction) constitute a process stream propelled into a process zone, which in the Microfluidizer® is called the Interaction Chamber. The product to be treated is inducted into the pump, and then forced out. The priming valve of the Microfluidizer® purges air out of the pump. Once the pump is filled with product, the priming valve is closed and the product is forced through the interaction chamber. The geometry of the interaction chamber produces powerful forces of sheer, impact, and cavitation which are responsible for particle size reduction. Specifically, inside the interaction chamber, the pressurized product is split into tow streams and accelerated to extremely high velocities. The formed jets are then directed toward each other and collide in the interaction zone. The resulting product has very fine and uniform particle or droplet size. The Microfluidizer® also provides a heat exchanger to allow cooling of the product.

4. Cryogenic Methodologies to Obtain Nanoparticulate Lipase Inhibitor Compositions

Another method of forming the desired nanoparticulate lipase inhibitor, such as orlistat, composition is by spray freezing into liquid (SFL). This technology comprises an organic or organoaqueous solution of lipase inhibitor with stabilizers, which is injected into a cryogenic liquid, such as liquid nitrogen. The droplets of the lipase inhibitor solution freeze at a rate sufficient to minimize crystallization and particle growth, thus formulating nanostructured lipase inhibitor particles. Depending on the choice of solvent system and processing conditions, the nanoparticulate lipase inhibitor particles can have varying particle morphology. In the isolation step, the nitrogen and solvent are removed under conditions that avoid agglomeration or ripening of the lipase inhibitor particles.

As a complementary technology to SFL, ultra rapid freezing (URF) may also be used to create equivalent nanostructured lipase inhibitor particles with greatly enhanced surface area. URF comprises an organic or organoaqueous solution of lipase inhibitor with stabilizers onto a cryogenic substrate.

5. Emulsion Methodologies to Obtain Nanoparticulate Lipase Inhibitor Compositions

Another method of forming the desired nanoparticulate lipase inhibitor, such as orlistat, composition is by template emulsion. Template emulsion creates nanostructured lipase inhibitor particles with controlled particle size distribution and rapid dissolution performance. The method comprises an oil-in-water emulsion that is prepared, then swelled with a non-aqueous solution comprising the lipase inhibitor and stabilizers. The particle size distribution of the lipase inhibitor particles is a direct result of the size of the emulsion droplets prior to loading with the lipase inhibitor, a property which can be controlled and optimized in this process. Furthermore, through selected use of solvents and stabilizers, emulsion stability is achieved with no or suppressed Ostwald ripening. Subsequently, the solvent and water are removed, and the stabilized nanostructured lipase inhibitor particles are recovered. Various lipase inhibitor particles morphologies can be achieved by appropriate control of processing conditions.

6. Supercritical Fluid Techniques Used to Obtain Nanoparticulat Lipase Inhibitor Compositions

Published International Patent Application No. WO 97/144407 to Pace et al., published Apr. 24, 1997, discloses particles of water insoluble biologically active compounds with an average size of 100 nm to 300 nm that are prepared by dissolving the compound in a solution and then spraying the solution into compressed gas, liquid or supercritical fluid in the presence of appropriate surface modifiers.

D. Methods of Using the Nanoparticulate Lipase Inhibitor Compositions of the Invention

The invention provides a method of rapidly increasing the amount of dissolved lipase inhibitor, such as orlistat, in a subject. Such a method comprises orally administering to a subject an effective amount of a composition comprising a lipase inhibitor. By rapidly increasing the dissolved amount at the site of action, more is available to bind to lipase than would be with a conventional dosage form. Therefore, a lower dose of nanoparticulate lipase inhibitor has the same efficacy as a higher dose of conventional, non-nanoparticulate lipase inhibitor.

The compositions of the invention are useful in treating obesity and related diseases. Obesity-related diseases include, but are not limited to, type II diabetes, high blood pressure, stroke, myocardial infarction, congestive heart failure, cancer, gallstones, gall bladder disease, gout, gouty arthritis, osteoarthritis, sleep apnea, and pickwickian syndrome.

The lipase inhibitor compounds of the invention may be administered to a subject via any conventional means including, but not limited to, orally, rectally, parenternally (e.g., intravenous, intramuscular, or subcutaneous), intracistemally, pulmonary, intravaginally, intraperitoneally, locally (e.g., powders, ointments or drops) or as a buccal or nasal spray. Oral administration is preferred. As used herein, the term “subject” is used to mean an animal, preferably a mammal, including a human or non-human. The terms patient and subject may be used interchangeably.

The nanoparticulate lipase inhibitor, such as orlistat, compositions may also comprise adjuvants such as preserving, wetting, emulsifying and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like.

Solid dosage forms for oral administration include, but are not limited to, capsules, tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to a lipase inhibitor, preferably orlistat, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Compositions suitable for parenternal injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, but eh maintenance of the required particle size in the case of dispersions, and by the use of surfactants.

Besides such inert diluents, the composition can also include adjuvants, such as wetting agents, emulsifying and suspending agents, sweetening, flavoring, and perfuming agents.

“Therapeutically effective amount” as used herein with respect to a lipase inhibitor dosage shall mean that dosage that provides the specific pharmacological response for which a lipase inhibitor is administered in a significant number of subjects in need of such treatment. It is emphasized that “therapeutically effective amount,” administered to a particular subject in a particular instance will not always be effective in treating the diseases described herein, even though such dosage is deemed a “therapeutically effective amount” by those skilled in the art. It is to be further understood that lipase inhibitor dosages are, in particular instances, measured as oral dosages, or with reference to drug levels as measured in blood.

One of ordinary skill will appreciate that effective amounts of a lipase inhibitor can be determined empirically and can be employed in pure form or, where such forms exist, in pharmaceutically acceptable salt, ester, or prodrug form. Actual dosage levels of a lipase inhibitor in the nanoparticulate compositions of the invention may be varied to obtain an amount of a lipase inhibitor that is effective to obtain a desired therapeutic response for a particular composition and method of administration. The selected dosage level therefore depends upon the desired therapeutic effect, the route of administration, the potency of the administered lipase inhibitor, the desired duration of treatment, and other factors.

Dosage unit compositions may contain such amounts of such submultiples thereof as may be used to make up the daily dose. It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

Thus, a precise pharmaceutically effective amount cannot be specified in advance and can be readily determined by the caregiver or clinician. Appropriate amounts can be determined by routine experimentation from animal models and human clinical studies.

The following examples are given to illustrate the present invention. It should be understood, however, that the spirit and scope of the invention is not to be limited to the specific conditions or details described in these examples but should only be limited by the scope of the claims that follow. All references identified herein, including U.S. patents, are hereby expressly incorporated by reference.

### EXPERIMENTAL EXAMPLES

25 different formulations of nanoparticulate orlistat were prepared using a NanoMill-01®, 10-ml chamber (NanoMill Systems, King of Prussia, Philadelphia, also described in U.S. Pat. No. 6,431,478), and PolyMill® 500, 500 micron polymeric attrition media (Dow Chemical Co.). The composition of each of the 25 formulations is shown in Table 6. The 25 formulations differed in composition (described in Table 6), or in mill speed and mill time used in the particle size reduction process (described in Table 7).

For the particle size reduction process, the one or more surface stabilizers were dissolved in water and orlistat was then added to this solution to form a mixture. The mixture and PolyMill® 500, 500 micron polymeric attrition media were loaded in the 10 mL chamber of the NanoMill® at an 89% media load for all formulations except formulation 12, which was loaded at 80%. The compositions were milled at the mill speed and for the time period shown in Table 7.

Following milling nanoparticulate orlistat particles were harvested using a 21, 29, or 29.5 gauge syringe. Particle size was determined using a Horiba LA-910 light scattering particle size analyzer both after milling, and after a 60 second sonication. Table 8 show the mean, mode and median particle size, as well as the D50, D90 and D95 particle sizes before and after sonication (“60s son”; “N” indicates no sonication, “Y” indicates 60 second sonication before particle size determination). The particle size measurement medium was water or Milli-Q water.

Processing comments and microscopic observations were noted for some of the formulations after preparation, as noted in Table 9. Microscopy was performed using either Leica DM5000B and Leica CTR 5000 light source, (Laboratory Instruments & Supplies (I) Ltd., Ashbourne, Colo. MEATH ROI) or by analyzing data from the Horiba LA-910 (Particular Sciences, Hatton Derbyshire, England).

Additionally, particle size was determined for nine of the formulations after storage for 14 days at 5° C., 25° C., and at 40° C. Stored particle size was determined both before and after sonication for 60 seconds; samples were tested in duplicate. Data is shown in Tables 10a and 10b. Table 10a shows the formulation number, the storage time, storage condition (temperature and relative humidity, “RH”), and the D50, D90 and D95 particle sizes. Table 10b again shows the formulation number, storage time and temperature (for reference), and lists statistics for particle size, including mean, mode and median. For each table 6-10, the formulation number (“Formulation Number” in Table 6; “No.” in Tables 7-10), which corresponds to the formulation provided in Table 6 and the milling parameters provided in Tables 7, is listed in the first column.

Formulation 11 was processed, sampled and analyzed, then further processed. The data in Tables 7-10 represents the final formulation.

The formulations and methods listed below are not intended to be limiting, rather to provide exemplary formulations and methods which may be useful in practicing the invention.

As shown in Table 6, various different surface stabilizers at different concentrations were evaluated to determine whether they could successfully stabilize a nanoparticulate orlistat composition, with “success” being defined as a resultant orlistat composition having a D50 of less than about 2 microns. For some compositions, more than one sample was prepared (i.e., composition Nos. 3, 6, 7, 8, 11, 12, 13, 15, 16, 17, 18, 19, 20, 21, 22, 23, 24, and 25). The different samples were prepared using different mill speeds and/or milling times (see Table 7).

TABLE NO. 6
Nanoparticulate orlistat formulations

| Formulation | |
|---|---|
| No. | Formulation |
| 1. | Orlistat, 5% (w/w) |
| | Pharmacoat ® 603, 2% (w/w) |
| | (hydroxypropyl methylcellulose) |
| | Deionised water, 93% (w/w) |
| 2. | Orlistat, 5% (w/w) |
| | Pharmacoat ® 603, 1.25% (w/w) |
| | (hydroxypropyl methylcellulose) |
| | Sodium Lauryl Sulfate, 0.05% (w/w) |
| | Deionised Water, 93.7% (w/w) |
| 3. | Orlistat, 5% (w/w) |
| | Pharmacoat ® 603, 2% (w/w) |
| | (hydroxypropyl methylcellulose) |
| | Deionised water, 93% (w/w) |
| 4. | Orlistat, 5% (w/w) |
| | HPC-SL, 1.5% (w/w) (hydroxypropyl Cellulose - |
| | Super Low viscosity) |
| | Deionised Water, 93.5% (w/w) |
| 5. | Orlistat, 5% (w/w) |
| | HPC-SL, 1.25% (w/w) (hydroxypropyl Cellulose - |
| | Super Low viscosity) |
| | Docusate Sodium, 0.05% (w/w) |
| | Deionised Water, 93.7% (w/w) |
| 6. | Orlistat, 5% (w/w) |
| | HPC-SL, 2% (w/w) (Hydroxypropyl Cellulose - |
| | Super Low viscosity) |
| | Deionised Water, 93% (w/w) |
| 7. | Orlistat, 5% (w/w) |
| | HPC-SL, 2% (w/w) (hydroxypropyl Cellulose - Super Low |
| | viscosity) |
| | Deionised Water, 93% (w/w) |
| 8. | Orlistat, 5% (w/w) |
| | HPC-SL, 1.25% (w/w) (hydroxypropyl Cellulose - |
| | Super Low viscosity) |
| | Docusate Sodium, 0.05% (w/w) |
| | Deionised Water, 93.7% (w/w) |
| 9. | Orlistat 5% (w/w) |
| | Plasdone ® S-630, 2% (w/w) (Copovidone K25-34) |
| | Deionised Water, 93% (w/w) |
| 10. | Orlistat 5% (w/w) |
| | Plasdone ® S-630, 1.5% (w/w) (Copovidone K25-34) |
| | Deionised Water, 93.5% (w/w) |
| 11. | Orlistat 5% (w/w) |
| | Plasdone ® S-630, 1.25% (w/w) (Copovidone K25-34) |
| | Docusate Sodium, 0.05% (w/w) |
| | Deionised Water, 93.7% (w/w) |
| 12. | Orlistat, 5% (w/w) |
| | Tween ® 80, 1.5% (w/w) (Polyoxyethylene Soribtan |
| | Fatty Acid Ester) |
| | Deionised Water, 93.5% (w/w) |
| 13. | Orlistat, 5% (w/w) |
| | Plasdone ® K-17, 1.25% (w/w) (Povidone K17) |
| | Benzalkonium Chloride, 0.05% (w/w) |
| | Deionised Water, 93.7% (w/w) |
| 14. | Orlistat, 5% (w/w) |
| | Plasdone ® K29/32, 1.5% (w/w) (Copovidone K25-34) |
| | Deionised water, 93.5% (w/w) |
| 15. | Orlistat, 5% (w/w) |
| | Pharmacoat ® 603, 1.25% (w/w) |
| | Sodium Lauryl Sulfate, 0.05% (w/w) |
| | Deionised Water, 93.7% (w/w) |
| 16. | Orlistat, 5% (w/w) |
| | Pluronic ® F108, 1.00% (w/w) |
| | Tween ® 80, 1.00% (w/w) |
| | Deionised Water, 93% (w/w) |
| 17. | Orlistat, 5% (w/w) |
| | Plasdone ® C-15, 1.25% (w/w) |
| | Deoxycholate acid, Sodium salt, 0.05% (w/w) |
| | Deionised Water, 93.7% (w/w) |
| 18. | Orlistat, 5% (w/w) |
| | Sodium Lauryl Sulfate, 0.1% (w/w) |
| | Deionised Water, 94.9% (w/w) |
| 19. | Orlistat, 5% (w/w) |
| | Docusate Sodium, 0.1% (w/w) |
| | Deionised Water, 94.9% (w/w) |
| 20. | Orlistat, 5% (w/w) |
| | Lutrol ® F127, 1.5% (w/w) (Poloxamer 407) |
| | Deionised Water, 93.5% (w/w) |
| 21. | Orlistat, 5% (w/w) |
| | Pluronic ® F108, 1.5% (w/w) |
| | Deionised Water, 93.5% (w/w) |
| 22. | Orlistat, 5% (w/w) |
| | Plasdone ® S-630, 1.25% (w/w) |
| | Docusate Sodium, 0.05% (w/w) |
| | Deionised Water, 93.7% (w/w) |
| 23. | Orlistat, 5% (w/w) |
| | Plasdone ® K29/32, 2% (w/w) |
| | Deionised Water, 93% (w/w) |
| 24. | Orlistat, 5% (w/w) |
| | Tween ® 80, 1.5% (w/w) |
| | Lecithin, 0.1% (w/w) |
| | Deionised Water, 93.4% (w/w) |
| 25. | Orlistat, 5% (w/w) |
| | Plasdone ® K29/32, 2% (w/w) |

TABLE 7
Mill Speed (RPM) and Mill Time/Min

| No. | Mill Speed RPM | Mill Time/Min |
|---|---|---|
| 1. | 2500 | 60 |
| 2. | 2500 | 60 |
| 3. | 4500 | 30 |
| | 2500 | 90 |
| | Sample 3 was milled for 30 min. at 4500 rpm and then 90 min. | |
| | at 2500 rpm, continuously. | |
| 4. | 2500 | 60 |
| 5. | 5500 | 60 |
| 6. | 2500 | 60 |
| | 2500 | 60 |
| | 4500 | 30 |
| | Sample 6a in Table 8 was processed at 2500 rpm for 60 min., | |
| | sampled and analyzed, and then processed for a further 60 min. | |
| | Data in Table 8 represents total milling time of 2500 rpm for | |
| | 120 min. | |
| | Sample 6b in Table 8 was further processed for 30 min. at | |
| | 4500 rpm. | |
| 7. | 4500 | 30 |
| | 2500 | 60 |
| | Sample 7 was milled for 30 min. at 4500 rpm and then 60 min. | |
| | at 2500 rpm, continuously. | |
| 8. | 4500 | 30 |
| | 2500 | 60 |
| | Sample 8 was milled for 30 min. at 4500 rpm and then 60 min. | |
| | at 2500 rpm, continuously. | |
| 9. | 4000 | 60 |
| 10. | 2500 | 60 |
| 11. | 2500 | 60 |
| | 2500 | 30 |
| | Sample 11 was processed at 2500 rpm for 60 min., sampled and | |
| | analyzed, and then processed for a further 30 min. Data | |
| | represents total milling time of 2500 rpm for 90 min. | |
| 12. | 2500 | 60 |
| | 5500 | 60 |
| | Sample 12 (both 12a and 12b in Table 8) was milled for 60 min. | |
| | at 2500 rpm and then 60 min. at 5500 rpm. | |
| 13. | 5500 | 10 |
| | 2500 | 30 |
| | Sample 13 was milled for 10 min. at 5500 rpm and then 30 min. | |
| | at 2500 rpm, continuously. | |
| 14. | 2500 | 60 |
| 15. | 4500 | 45 |
| | 2500 | 90 |
| | Sample 15 was milled for 45 min. at 4500 rpm and then 90 min. | |
| | at 2500 rpm, continuously. | |
| 16. | 1500 | 60 |
| | 4500 | 30 |
| | 2500 | 90 |
| | Sample 16 was milled for 10 min. at 1500 rpm, then 30 min. at | |
| | 4500 rpm, and finally 90 min. at 2500 rpm, continuously. | |
| 17. | 4500 | 45 |
| | 2500 | 90 |
| | Sample 17 was milled for 45 min. at 4500 rpm, then 90 min. at | |
| | 2500 rpm, continuously. | |
| 18. | 900 | 10 |
| | 4500 | 60 |
| | 2500 | 60 |
| | Sample 18 was milled for 10 min. at 900 rpm, then 60 min. at | |
| | 4500 rpm, and finally 60 min. at 2500 rpm, continuously. | |
| 19. | 900 | 10 |
| | 4500 | 60 |
| | 2500 | 60 |
| | Sample 19 was milled for 10 min. at 900 rpm, then 60 min. at | |
| | 4500 rpm, and finally 60 min. at 2500 rpm, continuously. | |
| 20. | 4500 | 30 |
| | 2500 | 90 |
| | Sample 20 was milled for 30 min. at 4500 rpm, then 90 min. at | |
| | 2500 rpm, continuously. | |
| 21. | 4500 | 30 |
| | 2500 | 90 |
| | Sample 21 was milled for 30 min. at 4500 rpm, then 90 min. at | |
| | 2500 rpm, continuously. | |
| 22. | 4500 | 30 |
| | 2500 | 90 |
| | Sample 22 was milled for 30 min. at 4500 rpm, then 90 min. at | |
| | 2500 rpm, continuously. | |
| 23. | 4500 | 30 |
| | 2500 | 90 |
| | 4500 | 45 |
| | Sample 23a in Table 8 was milled for 30 min. at 4500 rpm and | |
| | then 90 min. at 2500 rpm, continuously. | |
| | Sample 23b in Table 8 was further milled for 45 min. at | |
| | 4500 rpm. | |
| 24. | 4500 | 30 |
| | 2500 | 90 |
| | Sample 24 was milled for 30 min. at 4500 rpm and then 90 min. | |
| | at 2500 rpm, continuously. | |
| 25. | 4500 | 30 |
| | 2500 | 90 |

Sample 25 was milled for 30 min. at 4500 rpm and then 90 min.
at 2500 rpm, continuously.

TABLE 8
Particle size of formulations, with and without 60 second sonication

| | Mean/ | D50/ | D90/ | D95/ | Mode/ | Median/ | 60 s | |
|---|---|---|---|---|---|---|---|---|
| No. | nm | nm | nm | nm | nm | nm | son | Lamp % |
| 1. | 360 | 311 | 521 | 678 | 314 | 311 | N | 80.6 |
| | 347 | 309 | 504 | 632 | 313 | 309 | Y | 80.8 |
| 2. | 196 | 185 | 258 | 295 | 183 | 185 | N | 81.2 |
| | 195 | 185 | 257 | 294 | 183 | 185 | Y | 81.2 |
| 3. | 436 | 339 | 658 | 1009 | 318 | 339 | N | 81.8 |
| | 390 | 334 | 599 | 795 | 317 | 334 | Y | 82.3 |
| 4. | 331 | 312 | 474 | 547 | 315 | 312 | N | 80.3 |
| | 328 | 309 | 468 | 541 | 315 | 309 | Y | 80.6 |
| 5. | 724 | 321 | 690 | 2037 | 314 | 321 | N | 79.6 |
| | 371 | 313 | 561 | 761 | 313 | 313 | Y | 79.8 |
| 6a. | 327 | 307 | 467 | 540 | 314 | 307 | N | 82.8 |
| | 325 | 306 | 465 | 537 | 314 | 306 | Y | 83.0 |
| 6b. | 2640 | 454 | 7656 | 15387 | 361 | 454 | N | 79.6 |
| | 738 | 418 | 1647 | 2516 | 360 | 418 | Y | 79.8 |
| 7. | 419 | 330 | 639 | 971 | 316 | 330 | N | 81.3 |
| | 438 | 331 | 671 | 1100 | 316 | 331 | Y | 81.3 |
| 8. | 232 | 174 | 318 | 537 | 162 | 174 | N | 80.7 |
| | 263 | 217 | 371 | 532 | 209 | 217 | Y | 80.9 |
| 9. | 1975 | 343 | 4845 | 11605 | 280 | 343 | N | 79.0 |
| | 715 | 327 | 1871 | 3031 | 280 | 327 | Y | 79.3 |
| 10. | 15789 | 433 | 48226 | 96507 | 279 | 433 | N | 79.5 |
| | 1594 | 310 | 5555 | 9791 | 278 | 310 | Y | 79.8 |
| 11. | 179 | 161 | 228 | 274 | 160 | 161 | N | 79.5 |
| | 175 | 160 | 225 | 264 | 160 | 160 | Y | 79.6 |
| 12a. | 183 | 174 | 235 | 261 | 165 | 174 | N | 85.4 |
| | 181* | 173* | 233* | 258* | 165* | 173* | N* | 84.2 |
| | 174 | 168 | 223 | 247 | 163 | 168 | Y | 84.8 |
| 12b. | 31210 | 25255 | 58356 | 80490 | 31837 | 25255 | N | 80.5 |
| | 2891 | 1183 | 7909 | 11433 | 317 | 1183 | Y | 77.9 |
| 13. | 30877 | 23733 | 50360 | 69350 | 24428 | 23733 | N | 91.4 |
| | 17316 | 10419 | 41231 | 51868 | 27980 | 10419 | Y | 89.7 |
| 14. | 3775 | 373 | 14733 | 22185 | 316 | 373 | N | 82.7 |
| | 1213 | 342 | 2378 | 7158 | 316 | 342 | Y | 82.8 |
| | 9611* | 579* | 34312* | 48683* | 316* | 579* | N | 80.7 |
| | 7150* | 498* | 24407* | 37552* | 316* | 498* | Y | 80.7 |
| 15. | 400 | 289 | 537 | 1017 | 277 | 289 | N | 79.5 |
| | 340 | 286 | 485 | 673 | 277 | 286 | Y | 79.7 |
| 16. | 176 | 164 | 225 | 261 | 161 | 164 | N | 79.9 |
| | 168 | 161 | 219 | 246 | 160 | 161 | Y | 80.4 |
| 17. | 4740 | 855 | 15463 | 22189 | 414 | 855 | N | 80.4 |
| | 4648 | 775 | 14211 | 27454 | 365 | 775 | Y | 80.7 |
| 18. | 7957 | 377 | 23593 | 29450 | 277 | 377 | N | 86.6 |
| | 858 | 275 | 468 | 6179 | 274 | 275 | Y | 86.4 |
| 19. | 1492 | 300 | 2909 | 10340 | 278 | 300 | N | 80.3 |
| | 987 | 279 | 468 | 4524 | 276 | 279 | Y | 80.7 |
| 20. | 347 | 205 | 436 | 1330 | 188 | 205 | N | 79.5 |
| | 265 | 192 | 314 | 617 | 184 | 192 | Y | 80.3 |
| 21. | 649 | 296 | 1907 | 2925 | 276 | 296 | N | 81.4 |
| | 580 | 292 | 1487 | 2628 | 276 | 292 | Y | 82.0 |
| 22. | 498 | 297 | 1027 | 1888 | 277 | 297 | N | 79.4 |
| | 479 | 293 | 956 | 1800 | 276 | 293 | Y | 79.4 |
| 23a. | 798 | 300 | 2544 | 3631 | 276 | 300 | N | 79.7 |
| | 380 | 271 | 462 | 1153 | 273 | 271 | Y | 81.2 |
| 23b. | 21143 | 19673 | 41250 | 50111 | 24473 | 19673 | N | 78.0 |
| | 2946 | 2432 | 6121 | 7835 | 2782 | 2432 | Y | 69.6 |
| 24. | 395 | 293 | 542 | 1116 | 278 | 293 | N | 81.6 |
| | 317 | 277 | 428 | 537 | 276 | 277 | Y | 82.1 |
| 25. | 417 | 141 | 218 | 884 | 140 | 141 | N | 81.0 |
| | 321 | 137 | 197 | 247 | 140 | 137 | Y | 81.7 |

*Repeat measurement.

As shown in Table 8, not all combinations of surface stabilizer(s) and orlistat produced a stable nanoparticulate orlistat composition. Moreover, compositions demonstrating a significant difference in orlistat particle size following 60 seconds sonication likely contain agglomerates of orlistat particles. Such agglomerates are undesirable, as highly varying particle sizes, such as those present with nanoparticulate and agglomerated orlistat particles are present, can create highly variable absorption rates. Thus, it can be difficult to control individual dosages of a drug with such highly variable particle sizes.

TABLE 9
Processing comments and microscopy observations

| No. | Processing Comments | Microscopy Observation |
|---|---|---|
| 1. | API was extremely difficult | The sample looked very well dispersed |
| | to ‘wet in’ | with discrete orlistat nanoparticles clearly |
| | | visible. Brownian motion was also |
| | | clearly evident with no signs of orlistat |
| | | crystal growth. There was a reasonable |
| | | amount of “unmilled” orlistat particles, |
| | | which in some cases appeared “rod-like.” |
| | | There was also possibly some broken |
| | | media fragments. |
| 2. | Un-milled’ drug material | In general the sample appeared well |
| | present in milling chamber | dispersed with orlistat nanoparticles |
| | | clearly visible. Brownian motion was |
| | | also evident. However, there were larger |
| | | “un-milled” orlistat crystals present. |
| | | There was no sign of flocculation |
| 3. | | The sample looked very well dispersed |
| | | with orlistat nanoparticles visible; |
| | | however, the sample appeared less |
| | | populated with orlistat nanoparticles, this |
| | | maybe due to the presence of ‘rod’ shaped |
| | | particles. Brownian motion was also |
| | | clearly evident with no signs of orlistat |
| | | crystal growth. |
| 4. | No significant quantity of | The sample looked very well dispersed |
| | ‘un-milled’ drug. Although | with discrete nano-particulates clearly |
| | the material was extremely | visible. Brownian motion was also |
| | hard to harvest using the | clearly evident. There was signs of ‘un- |
| | syringe. | milled’ drug and small clusters of nanoparticulates. |
| | | The shape of the orlistat |
| | | nanoparticles and larger orlistat crystals |
| | | in some cases were “rod like”. |
| 5. | | The sample appeared well dispersed with |
| | | orlistat nanoparticulates clearly visible. |
| | | Brownian motion was also clearly |
| | | evident. Unmilled orlistat drug crystals |
| | | were observed throughout the sample |
| | | with isolated areas of localized |
| | | flocculation. |
| 6a. | “un-milled” drug crystals | Microscopy not performed on |
| | observed | “in process” sample. |
| 6b. | | Microscopy not performed on |
| | | “in process” sample. |
| 7. | | The sample appeared well dispersed with |
| | | orlistat nanoparticles clearly visible. |
| | | Brownian motion was also evident. |
| | | There was also evidence of ‘un-milled’ |
| | | drug crystals. Rod-like crystals were |
| | | observed which may be indicative of |
| | | crystal growth. There was very little sign |
| | | of flocculation. |
| 8. | | The sample showed clear evidence of |
| | | orlistat nanoparticles. Brownian motion |
| | | was also evident. However, there were |
| | | particulates of larger ‘un-milled’ drug |
| | | apparent. There was no sign of |
| | | flocculation or crystal growth. |
| 9. | | In general the sample appeared well |
| | | dispersed with orlistat nanoparticles |
| | | clearly visible. Brownian motion was |
| | | also evident. There were signs of larger |
| | | ‘un-milled’ drug crystals and some signs |
| | | of clusters of agglomerated drug crystal |
| 10. | | In general the sample appeared well |
| | | dispersed with orlistat nanoparticles |
| | | clearly visible. Brownian motion was |
| | | also evident. The majority of particles |
| | | were needle-like in shape. Larger |
| | | “unmilled” drug crystals were apparent |
| | | with signs of flocculation. |
| 11. | Significant amount of | In general the sample appeared well |
| | umilled drug particles were | dispersed with orlistat nanoparticles |
| | present in the chamber and | clearly visible. Brownian motion was |
| | on the agitator. | also evident. The majority of particles |
| | | were rod-like in shape which may be |
| | | indicative of crystal growth. Large |
| | | “unmilled” drug crystals were also |
| | | apparent. |
| 12a | Un-milled’ drug material | Microscopy not performed on “in |
| | present in milling chamber | process” sample. |
| 12b | | Microscopy not performed on “in |
| | | process” sample. |
| 13. | ‘Un-milled’ drug material | Microscopy was not performed. |
| | present in milling chamber | |
| 14. | | The sample appeared well dispersed with |
| | | orlistat nanoparticles clearly visible. |
| | | Brownian motion was also evident. |
| | | However, flocculation could be seen in a |
| | | number of areas, along with smaller |
| | | clusters of nanoparticles. |
| 15. | | The sample appeared well dispersed with |
| | | orlistat nanoparticles clearly visible. |
| | | Brownian motion was also evident. |
| | | There were no signs of flocculated drug |
| | | crystal. |
| 16. | A small quantity of ‘un- | The sample appeared well dispersed with |
| | milled’ drug material present | orlistat nanoparticles clearly visible. |
| | in milling chamber | Brownian motion was also evident. The |
| | | drug crystals are rod-like in shape and |
| | | demonstrate that the sample may be |
| | | susceptible to crystal growth. |
| 17. | | In general the sample appeared well |
| | | dispersed with orlistat nanoparticles |
| | | clearly visible. A large quantity of |
| | | unmilled drug crystals were present. |
| | | Brownian motion was also evident. The |
| | | sample showed regions of localized |
| | | flocculation. |
| 18. | Un-milled’ drug material | Microscopy was not performed. |
| | present on chamber cover | |
| | plate and near motor section. | |
| 19. | ‘Un-milled’ drug material | The sample appeared well dispersed with |
| | present in milling chamber | orlistat nanoparticles clearly evident. |
| | | Brownian motion was also clearly |
| | | evident with no signs of flocculation or |
| | | crystal growth. There were some larger |
| | | “unmilled” drug crystals present.. |
| 20. | ‘Un-milled’ drug material | In general the sample appeared well |
| | present in milling chamber | dispersed with orlistat nanoparticles |
| | | clearly visible. Brownian motion was |
| | | also evident. Evidence of ‘un-milled’ |
| | | drug crystals was observed along with |
| | | rod-like crystals indicating possible |
| | | crystal growth. Isolated areas of localized |
| | | flocculation were also observed. |
| 21. | Bi-modal distribution on | The sample appeared well dispersed with |
| | Horiba pre and post | no signs of flocculation or crystal |
| | sonication may also indicate | growth. Orlistat nanoparticles and |
| | “unmilled” drug. | slightly larger unmilled drug were |
| | | dispersed throughout. Brownian motion |
| | | was clearly evident. |
| 22. | ‘Un-milled’ drug material | In general the sample appeared well |
| | present in milling chamber | dispersed with orlistat nanoparticles |
| | | clearly visible. Brownian motion was |
| | | also evident. Evidence of ‘un-milled’ |
| | | drug crystals was observed along with |
| | | isolated areas of localized flocculation. |
| 23. | A significant amount of | Microscopy was not performed. |
| | unmilled drug particulates | |
| | were observed in the | |
| | chamber after the first | |
| | milling stage. | |
| 24. | | The sample appeared well dispersed with |
| | | orlistat nanoparticles clearly visible. |
| | | Brownian motion was also evident. |
| | | There did not appear to be any evidence |
| | | of aggregation in the form of flocculation |
| | | or crystal growth. |
| 25. | Unmilled drug particles were | The sample appeared well dispersed with |
| | observed in the milling | orlistat nanoparticles. Unmilled drug |
| | chamber. | crystals and signs of crystal growth were |
| | | also observed. Small areas of |
| | | flocculation were observed. Brownian |
| | | motion was clearly evident. |

For surface stabilizer compositions that did not successfully stabilize orlistat as shown in the tables above, different concentrations of orlistat and/or the surface stabilizers may produce a stable nanoparticulate orlistat composition.

TABLE 10a
Stability testing: particle size with and without 60 second
sonication

| | Storage | | | | | |
|---|---|---|---|---|---|---|
| | time | Storage | D50/ | D90/ | D95/ | |
| No. | (days) | Condition | nm | nm | nm | 60 s son |
| 2 | 0 | N/A | 185 | 258 | 295 | N |
| | 0 | N/A | 185 | 257 | 294 | Y |
| | 14 | 5° C. | 196 | 100516 | 136455 | N |
| | 14 | 5° C. | 208 | 8771 | 18960 | Y |
| | 14 | 25° C./60% RH | 170 | 72678 | 104171 | N |
| | 14 | 25° C./60% RH | 212 | 717 | 7838 | Y |
| 3 | 0 | N/A | 339 | 658 | 1009 | N |
| | 0 | N/A | 334 | 599 | 795 | Y |
| | 14 | 5° C. | 23999 | 139683 | 178902 | N |
| | 14 | 5° C. | 362 | 7376 | 13593 | Y |
| 6 | 0 | N/A | 174 | 318 | 537 | N |
| | 0 | N/A | 217 | 371 | 532 | Y |
| | 14 | 5° C. | 265 | 130336 | 174898 | N |
| | 14 | 5° C. | 216 | 10063 | 22230 | Y |
| | 14 | 25° C./60% RH | 170 | 84494 | 118656 | N |
| | 14 | 25° C./60% RH | 192 | 302 | 2102 | Y |
| 7 | 0 | N/A | 330 | 639 | 971 | N |
| | 0 | N/A | 331 | 671 | 1100 | Y |
| | 14 | 5° C. | 64261 | 175942 | 221539 | N |
| | 14 | 5° C. | 399 | 19307 | 26735 | Y |
| | 14 | 25° C./60% RH | 19086 | 78580 | 96406 | N |
| | 14 | 25° C./60% RH | 310 | 3568 | 7161 | Y |
| 8 | 0 | N/A | 174 | 318 | 537 | N |
| | 0 | N/A | 217 | 371 | 532 | Y |
| | 14 | 5° C. | 265 | 130336 | 174898 | N |
| | 14 | 5° C. | 216 | 10063 | 22230 | Y |
| | 14 | 25° C./60% RH | 170 | 84494 | 118656 | N |
| | 14 | 25° C./60% RH | 192 | 302 | 2102 | Y |
| | 14 | 40° C./60% RH | 392 | 532 | 577 | N |
| | 14 | 40° C./60% RH | 395 | 540 | 582 | Y |
| 15 | 0 | N/A | 289 | 537 | 1017 | N |
| | 0 | N/A | 286 | 485 | 673 | Y |
| | 14 | 5° C. | 4924 | 209398 | 266403 | N |
| | 14 | 5° C. | 311 | 12049 | 24135 | Y |
| | 14 | 25° C./60% RH | 189 | 58786 | 88122 | N |
| | 14 | 25° C./60% RH | 211 | 984 | 3870 | Y |
| | 14 | 40° C./75% RH | 383 | 543 | 596 | N |
| | 14 | 40° C./75% RH | 387 | 553 | 612 | Y |
| 16 | 0 | N/A | 164 | 225 | 261 | N |
| | 0 | N/A | 161 | 219 | 246 | Y |
| | 14 | 5° C. | 152 | 486 | 8500 | N |
| | 14 | 5° C. | 194 | 272 | 345 | Y |
| | 14 | 25° C./60% RH | 371 | 90884 | 120263 | N |
| | 14 | 25° C./60% RH | 284 | 485 | 2949 | Y |
| 20 | 0 | N/A | 205 | 436 | 1330 | N |
| | 0 | N/A | 192 | 314 | 617 | Y |
| | 14 | 5° C. | 167 | 24954 | 46361 | N |
| | 14 | 5° C. | 192 | 271 | 344 | Y |
| 21 | 0 | N/A | 296 | 1907 | 2925 | N |
| | 0 | N/A | 292 | 1487 | 2628 | Y |
| | 14 | 5° C. | 15805 | 106358 | 133797 | N |
| | 14 | 5° C. | 164 | 286 | 693 | Y |

TABLE 10b
Stability testing, con't: statistics and lamp %

| | Storage | | | | | | |
|---|---|---|---|---|---|---|---|
| | time | Storage | Mean/ | Mode/ | Median/ | | |
| No. | (days) | Condition | nm | nm | nm | 60 s son. | Lamp % |
| 2 | 0 | N/A | 196 | 183 | 185 | N | 81.2 |
| | 0 | N/A | 195 | 183 | 185 | Y | 81.2 |
| | 14 | 5° C. | 28076 | 159 | 196 | N | 82.8 |
| | 14 | 5° C. | 2439 | 188 | 208 | Y | 83.1 |
| | 14 | 25° C./60% RH | 16072 | 158 | 170 | N | 82.1 |
| | 14 | 25° C./60% RH | 1214 | 206 | 212 | Y | 82.4 |
| 3 | 0 | N/A | 436 | 318 | 339 | N | 81.8 |
| | 0 | N/A | 390 | 317 | 334 | Y | 82.3 |
| | 14 | 5° C. | 50732 | 316 | 23999 | N | 80.2 |
| | 14 | 5° C. | 2045 | 316 | 362 | Y | 80.5 |
| 6 | 0 | N/A | 232 | 162 | 174 | N | 80.7 |
| | 0 | N/A | 263 | 209 | 217 | Y | 80.9 |
| | 14 | 5° C. | 40976 | 162 | 265 | N | 79.5 |
| | 14 | 5° C. | 3110 | 205 | 216 | Y | 79.9 |
| | 14 | 25° C./60% RH | 22255 | 156 | 170 | N | 81.1 |
| | 14 | 25° C./60% RH | 506 | 184 | 192 | Y | 81.4 |
| 7 | 0 | N/A | 419 | 316 | 330 | N | 81.3 |
| | 0 | N/A | 438 | 316 | 331 | Y | 81.3 |
| | 14 | 5° C. | 76195 | 108058 | 64261 | N | 78.0 |
| | 14 | 5° C. | 5418 | 280 | 399 | Y | 78.0 |
| | 14 | 25° C./60% RH | 29931 | 277 | 19086 | N | 79.8 |
| | 14 | 25° C./60% RH | 1211 | 278 | 310 | Y | 80.0 |
| 8 | 0 | N/A | 232 | 162 | 174 | N | 80.7 |
| | 0 | N/A | 263 | 209 | 217 | Y | 80.9 |
| | 14 | 5° C. | 40976 | 162 | 265 | N | 79.5 |
| | 14 | 5° C. | 3110 | 205 | 216 | Y | 79.9 |
| | 14 | 25° C./60% RH | 22255 | 156 | 170 | N | 81.1 |
| | 14 | 25° C./60% RH | 506 | 184 | 192 | Y | 81.4 |
| | 14 | 40° C./75% RH | 399 | 410 | 392 | N | 108.5 |
| | 14 | 40° C./75% RH | 402 | 411 | 395 | Y | 110.0 |
| 15 | 0 | N/A | 400 | 277 | 289 | N | 79.5 |
| | 0 | N/A | 340 | 277 | 286 | Y | 79.7 |
| | 14 | 5° C. | 64545 | 276 | 4924 | N | 80.9 |
| | 14 | 5° C. | 3405 | 277 | 311 | Y | 81.2 |
| | 14 | 25° C./60% RH | 13556 | 161 | 189 | N | 80.0 |
| | 14 | 25° C./60% RH | 814 | 189 | 211 | Y | 80.6 |
| | 14 | 40° C./75% RH | 394 | 409 | 383 | N | 81.4 |
| | 14 | 40° C./75% RH | 398 | 410 | 387 | Y | 83.1 |
| 16 | 0 | N/A | 176 | 161 | 164 | N | 79.9 |
| | 0 | N/A | 168 | 160 | 161 | Y | 80.4 |
| | 14 | 5° C. | 1790 | 156 | 152 | N | 78.4 |
| | 14 | 5° C. | 219 | 180 | 194 | Y | 79.1 |
| | 14 | 25° C./60% RH | 22241 | 280 | 371 | N | 80.1 |
| | 14 | 25° C./60% RH | 651 | 277 | 284 | Y | 80.6 |
| 20 | 0 | N/A | 347 | 188 | 205 | N | 79.5 |
| | 0 | N/A | 265 | 184 | 192 | Y | 80.3 |
| | 14 | 5° C. | 6161 | 159 | 167 | N | 80.1 |
| | 14 | 5° C. | 219 | 185 | 192 | Y | 80.7 |
| 21 | 0 | N/A | 649 | 276 | 296 | N | 81.4 |
| | 0 | N/A | 580 | 276 | 292 | Y | 82.0 |
| | 14 | 5° C. | 38717 | 276 | 15805 | N | 80.0 |
| | 14 | 5° C. | 314 | 160 | 164 | Y | 80.3 |

It will be apparent to those skilled in the art that various modifications and variations can be made in the methods and compositions of the present inventions without departing from the spirit or scope of the invention. Thus, it is intended that the present invention cover the modification and variations of the invention provided they come within the scope of the appended claims and their equivalents.

What is claimed is:

1. A stable nanoparticulate lipase inhibitor composition comprising:
(a) particles of at least one lipase inhibitor having an effective average particle size of less than about 2000 nm; and
(b) at least one surface stabilizer.

2. The composition of claim 1, wherein the nanoparticulate lipase inhibitor is orlistat.

3. The composition of claim 1, wherein the nanoparticulate lipase inhibitor particle is selected from the group consisting of a crystalline phase, an amorphous phase, a semi-crystalline phase, a semi amorphous phase, and mixtures thereof.

4. The composition of claim 1, wherein the effective average particle size of the nanoparticulate lipase inhibitor particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

5. The composition of claim 1, in which the lipase inhibitor particles possess an enhanced binding affinity for lipase in the lumen of the stomach and small intestine allowing for the administration of dosage amounts of less than about 375 mg daily to have the same efficacy of dosage amounts of about 375 mg daily of a conventional, non-nanoparticulate composition of the same lipase inhibitor.

6. The composition of claim 1, wherein the composition is formulated:
(a) into a dosage form selected from the group consisting of tablets, capsules, sachets, solutions, liquid dispersions, gels, aerosols, ointments, creams;
(b) into a dosage form selected from the group consisting of controlled release formulations, fast melt formulations, lyophilized formulations, delayed release formulations, extended release formulations, pulsatile release formulations, and mixed immediate release and controlled release formulations;
(c) for administration via a method selected from the group consisting of parenteral injection, oral administration, aerosol administration, vaginal, nasal, rectal, otic, ocular, local, buccal, intracisternal, intraperitoneal, and topical administration; or
(d) any combination thereof.

7. The composition of claim 6, wherein the composition further comprises one or more pharmaceutically acceptable excipients, carriers, or a combination thereof.

8. The composition of claim 1, wherein:
(a) the lipase inhibitor is present in an amount consisting of from about 99.5% to about 0.001%, from about 95% to about 0.1%, and from about 90% to about 0.5%, by weight, based on the total combined weight of the lipase inhibitor and at least one surface stabilizer, not including other excipients;
(b) the at least one surface stabilizer is present in an amount of from about 0.5% to about 99.999% by weight, from about 5.0% to about 99.9% by weight, and from about 10% to about 99.5% by weight, based on the total combined dry weight of the lipase inhibitor and at least one surface stabilizer, not including other excipients; or
(c) a combination thereof.

9. The composition of claim 1, wherein the surface stabilizer is selected from the group consisting of a non-ionic surface stabilizer, an anionic surface stabilizer, a cationic surface stabilizer, a zwitterionic surface stabilizer, and an ionic surface stabilizer.

10. The composition of claim 1, wherein the surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctyl sodium sulfosuccinate (also known as docusate sodium), dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, lysozyme, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipids, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C12-15dimethyl hydroxyethyl ammonium chloride, C12-15dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)4 ammonium chloride, lauryl dimethyl (ethenoxy)4 ammonium bromide, N-alkyl (C12-18)dimethylbenzyl ammonium chloride, N-alkyl (C14-18)dimethyl-benzyl ammonium chloride, N-tetradecylidmethylbenzyl ammonium chloride monohydrate, dimethyl didecyl ammonium chloride, N-alkyl and (C12-14) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C12-14) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C12 trimethyl ammonium bromides, C15 trimethyl ammonium bromides, C17 trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar.

11. The composition of claim 1, additionally comprising one or more active agents useful for the treatment of obesity and related diseases.

12. The composition of claim 11, wherein the one or more active agents is selected from the group consisting of anti-obesity agents, appetite suppressants, anti-diabetic agents, anti-hyperlipidemia agents, hypolipidemic agents, hypocholesterolemic agents, lipid-modulating agents, cholesterol-lowering agents, lipid-lowering agents, anti-hypertensive agents, agents used to treat sleep disorders, agents used to treat substance abuse and addictive disorders, anti-anxiety agents, anti-depressants, anti-psychotic agents, cognition enhancing agents, agents used to treat cognitive disorders, agents used to treat Alzheimer's disease, agents used to treat Parkinson's disease, anti-inflammatory agents, agents used to treat neurodegeneration, agents used to treat arteriosclerosis, agents used to treat respiratory conditions, agents used to treat bowel disorders, cardiac glycosides, and anti-tumor agents.

13. A method of preparing a nanoparticulate lipase inhibitor comprising contacting particles of a lipase inhibitor with at least one surface stabilizer for a time and under conditions sufficient to provide a nanoparticulate lipase inhibitor composition having an effective average particle size of less than about 2000 nm.

14. The method of claim 13, wherein the lipase inhibitor is orlistat.

15. The method of claim 13, wherein contacting comprising grinding, wet grinding, homogenization, precipitation, or super critical fluid particle generation.

16. The method of claim 13, wherein the effective average particle size of the nanoparticulate lipase inhibitor particles is selected from the group consisting of less than about 1900 nm, less than about 1800 nm, less than about 1700 nm, less than about 1600 nm, less than about 1500 nm, less than about 1000 nm, less than about 1400 nm, less than about 1300 nm, less than about 1200 nm, less than about 1100 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

17. A method of treatment of obesity or a related disease comprising the administration of a nanoparticulate lipase inhibitor composition comprising:
(a) particles of at least one lipase inhibitor having an effective average particle size of less than about 2000 nm; and
(b) at least one surface stabilizer.

18. The method of claim 17, wherein the lipase inhibitor is orlistat.

19. The method of claim 17, wherein the obesity-related disease is selected from the group consisting of type II diabetes, high blood pressure, stroke, myocardial infarction, congestive heart failure, cancer, gallstones, gall bladder disease, gout, gouty arthritis, osteoarthritis, sleep apnea, and pickwickian syndrome.

20. The method of claim 17, wherein the effective average particle size of the nanoparticulate lipase inhibitor particles is selected from the group consisting of less than about 1900 nm, less than about 1800 nm, less than about 1700 nm, less than about 1600 nm, less than about 1500 nm, less than about 1000 nm, less than about 1400 nm, less than about 1300 nm, less than about 1200 nm, less than about 1100 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.
